(19) United States
(12) Patent Application Publication
(10) Pub. No.: US 2025/0057445 A1
(43) Pub. Date: Feb. 20, 2025

(54) EVALUATION DEVICE, EVALUATION METHOD, AND RECORDING MEDIUM

(71) Applicant: NEC Corporation, Minato-ku, Tokyo (JP)

(72) Inventors: Zhenwei WANG, Tokyo (JP); Kenichiro FUKUSHI, Tokyo (JP); Chenhui HUANG, Tokyo (JP); Hiroshi KAJITANI, Tokyo (JP); Fumiyuki NIHEY, Tokyo (JP)

(73) Assignees: NEC Corporation, Minato-ku, Tokyo (JP); NEC Corporation, (omitted)

(21) Appl. No.: 18/724,271

(22) PCT Filed: Jan. 11, 2022
(86) PCT No.: PCT/JP2022/000463

§ 371 Date: Jun. 26, 2024

(51) Int. Cl. A61B 5/11; A61B 5/00; G16H 50/30

(52) U.S. Cl. CPC A61B 5/1116; A61B 5/7282; G16H 50/30; A61B 2505/09

(57) ABSTRACT

Provided is an evaluation device that includes an acquisition unit that acquires position data pertaining to a plurality of measurement target sites established in an evaluation target segment of a body, a calculation unit that fits a circle to the plurality of measurement target sites and calculates a value of a diameter of the fitted circle, an evaluation unit that evaluates flexibility of the evaluation target segment according to the calculated value of the diameter of the circle, and an output unit that outputs an evaluation result relating to the flexibility of the evaluation target segment.

### TECHNICAL FIELD

The present disclosure relates to an evaluation device and the like for evaluating flexibility of a body.

### BACKGROUND ART

An item for measuring flexibility around a back, such as uniform extension/bending of a spinal column, is one of evaluation items of a motor function. Regarding the measurement of the flexibility around the back, a method for correctly evaluating the flexibility has not been established. The measurement of the flexibility around the back is often performed based on the rules of thumb of the trainer.

PTL 1 discloses a motor function evaluation device that evaluates a function of a part of a body by using information on curvature of a trajectory relating to the part of the body. The device of PTL 1 acquires curvature-related information relating to curvature from a plurality of pieces of position information indicating a position of a part of a body in a case where an evaluated person moves the part of the body. The device of PTL 1 evaluates the function of the part of the body by using the acquired curvature-related information.

PTL 2 discloses a measurement device that measures a load applied to an intervertebral disc. The device of PTL 2 images the back of a subject who is moving and measures the movement of a vertebra constituting a spinal column. The device of PTL 2 calculates the deformation of the spinal column based on the measured movement of the vertebra. The device of PTL 2 estimates the load applied to the intervertebral disc based on the calculated deformation of the spinal column.

### CITATION LIST

### Patent Literature

PTL 1: JP 2009-285273 A
PTL 2: JP 2010-214098 A

### SUMMARY OF INVENTION

### Technical Problem

In the method of PTL 1, the function of an upper limb can be evaluated based on the curvature of the trajectory of the upper limb. However, the method of PTL 1 cannot evaluate the flexibility of the body.

In the method of PTL 2, the deformation of the spinal column can be calculated based on the movement of the vertebra constituting the spinal column. However, PTL 2 does not disclose evaluating the flexibility of the spinal column.

An object of the present disclosure is to provide an evaluation device and the like capable of evaluating flexibility of a body.

### Solution to Problem

An evaluation device according to one aspect of the present disclosure includes an acquisition unit that acquires position data pertaining to a plurality of measurement target sites established in an evaluation target segment of a body, a calculation unit that fits a circle to the plurality of measurement target sites and calculates a value of a diameter of the fitted circle, an evaluation unit that evaluates flexibility of the evaluation target segment according to the calculated value of the diameter of the circle, and an output unit that outputs an evaluation result relating to the flexibility of the evaluation target segment.

An evaluation method according to one aspect of the present disclosure acquires position data pertaining to a plurality of measurement target sites established in an evaluation target segment of a body, fits a circle to the plurality of measurement target sites and calculates a value of a diameter of the fitted circle, evaluates flexibility of the evaluation target segment according to the calculated value of the diameter of the circle, and outputs an evaluation result relating to the flexibility of the evaluation target segment.

A program according to one aspect of the present disclosure causes a computer to execute a process of acquiring position data pertaining to a plurality of measurement target sites established in an evaluation target segment of a body, a process of fitting a circle to the plurality of measurement target sites and calculating a value of a diameter of the fitted circle, a process of evaluating flexibility of the evaluation target segment according to the calculated value of the diameter of the circle, and a process of outputting an evaluation result relating to the flexibility of the evaluation target segment.

### Advantageous Effects of Invention

According to the present disclosure, it is possible to provide an evaluation device and the like capable of evaluating flexibility of a body.

### BRIEF DESCRIPTION OF DRAWINGS

FIG. 1 is a block diagram illustrating an example of a configuration of an evaluation device according to a first example embodiment.

FIG. 2 is a conceptual diagram for explaining an example (extension) of a posture to be evaluated by the evaluation device according to the first example embodiment.

FIG. 3 is a conceptual diagram for explaining another example (forward bending) of the posture to be evaluated by the evaluation device according to the first example embodiment.

FIG. 4 is a conceptual diagram for explaining an example in which circle fitting is performed on a posture in which a subject to be evaluated by the evaluation device according to the first example embodiment extends.

FIG. 5 is a conceptual diagram for explaining an example in which circle fitting is performed on a posture in which the subject to be evaluated by the evaluation device according to the first example embodiment bends forward.

FIG. 6 is a conceptual diagram for explaining determination of flexibility of a body by the evaluation device according to the first example embodiment.

FIG. 7 is a flowchart for explaining an example of an operation of the evaluation device according to the first example embodiment.

FIG. 8 is a flowchart for explaining an example of circle fitting processing by the evaluation device according to the first example embodiment.

FIG. 9 is a conceptual diagram for explaining an application example of the evaluation device according to the first example embodiment.

FIG. 10 is a block diagram illustrating an example of a configuration of an evaluation device according to a second example embodiment.

FIG. 11 is a conceptual diagram for explaining an example in which ellipse fitting is performed on a posture in which a subject to be evaluated by the evaluation device according to the second example embodiment extends.

FIG. 12 is a conceptual diagram for explaining a determination criterion of uniformity in bending of the body by the evaluation device according to the second example embodiment.

FIG. 13 is a flowchart for explaining an example of an operation of the evaluation device according to the second example embodiment.

FIG. 14 is a flowchart for explaining an example of ellipse fitting processing by the evaluation device according to the second example embodiment.

FIG. 15 is a conceptual diagram for explaining an application example of the evaluation device according to the second example embodiment.

FIG. 16 is a conceptual diagram for explaining an application example of the evaluation device according to the second example embodiment.

FIG. 17 is a block diagram illustrating an example of a configuration of an evaluation device according to a third example embodiment.

FIG. 18 is a block diagram illustrating an example of a hardware configuration that executes control and processing according to each example embodiment.

### EXAMPLE EMBODIMENT

Hereinafter, an example embodiment of the present invention will be described with reference to the drawings. However, the example embodiment described below have technically preferable limitations for carrying out the present invention, but the scope of the invention is not limited to the following. In all the drawings used in the following description of the following example embodiment, the same reference numerals are given to the same parts unless there is a particular reason. In the following example embodiment, repeated description of similar configurations and operations may be omitted.

### First Example Embodiment

First, an evaluation device according to a first example embodiment will be described with reference to the drawings. The evaluation device of the present example embodiment evaluates the flexibility of a body based on position information pertaining to a plurality of measurement target sites set in the body. In the present example embodiment, an example of evaluating the flexibility of an upper body in a posture in which the body is bent backward while standing (extended posture) will be described. A method of the present example embodiment can also be applied to a posture in which the body is bent forward in the standing position (forward bending posture) and a posture in which the body is bent sideways while standing (side bending posture). In addition, the method of the present example embodiment can also be applied to a posture in which the body is bent in the state of being seated on a floor or a posture in which the upper body is bent in a prone position.

### (Configuration)

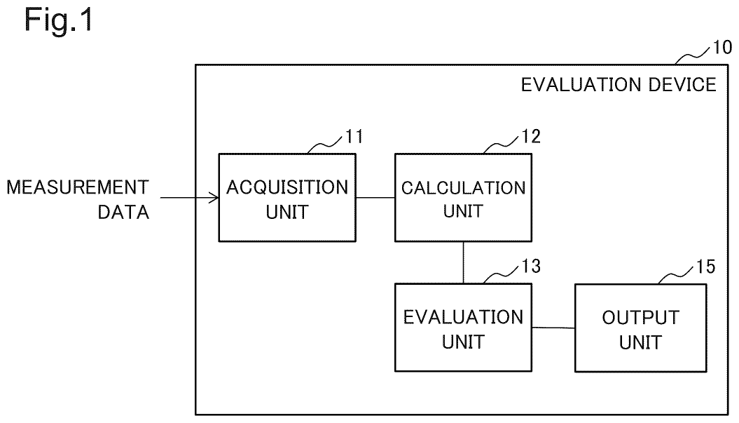

FIG. 1 is a block diagram for explaining an example of a configuration of an evaluation device 10 according to the present example embodiment. The evaluation device 10 includes an acquisition unit 11, a calculation unit 12, an evaluation unit 13, and an output unit 15.

The acquisition unit 11 acquires measurement data including the position information pertaining to the plurality of measurement target sites set in the body. For example, the acquisition unit 11 acquires position information of a marker installed at the measurement target site of the body of the subject. At least three markers are installed. The position information of the marker is measured by using an image captured by a camera. For example, when a sensor capable of measuring the position information is mounted on the marker, the position of the measurement target site can be specified according to the position information measured by the sensor. For example, the marker is installed at the position of the back, head, hand, knee, or ankle of the subject. When the markers are installed at the positions of the back, head, hand, knee, and ankle, the overall flexibility of the body of the subject can be evaluated. For example, a plurality of markers is installed along the spinal column of the subject. When a plurality of markers is installed along the spinal column, the flexibility of the spinal column can be evaluated.

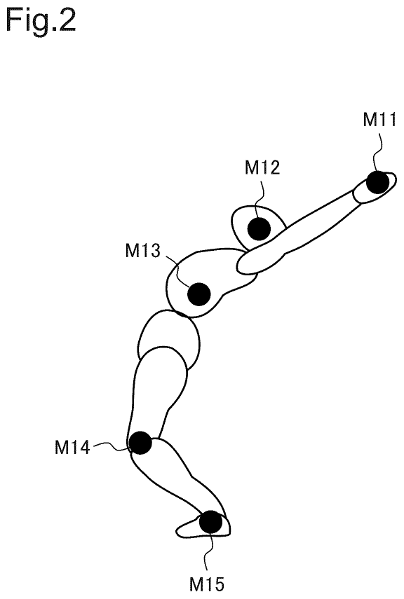

FIG. 2 is a conceptual diagram illustrating an example in which the markers are installed at the positions of the hand, head, spine, knee, and ankle of the subject. FIG. 2 illustrates a posture (extension) in which the upper body is bent backward. In FIG. 2, the positions of the hand, head, spine, knee, and ankle of the subject are denoted as M11, M12, M13, M14, and M15, respectively. It is sufficient if the marker is installed according to an evaluation target segment for flexibility. In a case where the evaluation target segment for flexibility is the spinal column, it is sufficient if a plurality of markers is installed along the spinal column.

For example, the acquisition unit 11 may acquire the position information pertaining to the measurement target site detected from the image of the subject in which no marker is installed on the body. For example, the measurement target sites such as the hand, the head, the spine, the knee, and the ankle can be extracted according to features detected from the image. The position of the measurement target site may be measurement data measured by general-purpose pose analysis software. For example, the position of the measurement target site may be estimated by using an image captured by a three-dimensional camera used for motion analysis. For example, the position of the measurement target site may be estimated by applying, to a skeleton model, physical quantities such as acceleration and angular velocity measured by an inertial measurement device installed in the measurement target site.

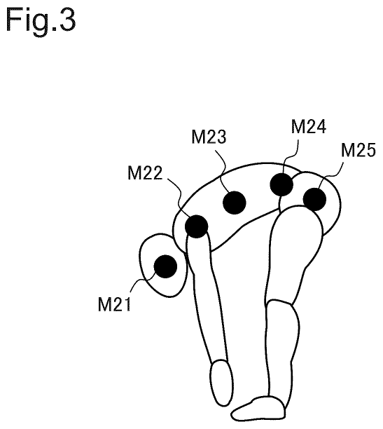

The acquisition unit 11 may acquire measurement data relating to a posture other than the extension. FIG. 3 is a conceptual diagram illustrating an example of a posture in which an upper body is bent forward (forward bending). FIG. 3 is a conceptual diagram illustrating an example in which markers are installed at the positions of the head, shoulder, spine, waist, and buttock of the subject. In FIG. 2, the positions of the head, shoulder, spine, waist, and buttock of the subject are denoted as M21, M22, M23, M24, and M25, respectively. Measurement data relating to a posture in which the body is bent sideways (side bending) can also be measured at the position of the marker in FIG. 3. In a case where the evaluation target segment for flexibility is the spinal column, it is sufficient if a plurality of markers is installed along the spinal column. For example, the evaluation target segment may be set to a cervical spine, a thoracic spine, or a lumbar spine constituting the spinal column. In addition, as in the present example embodiment, the evaluation target segment may be set in the entire body including an arm and a leg.

The calculation unit 12 acquires, from the acquisition unit 11, the position information pertaining to the plurality of measurement target sites relating to the subject. The calculation unit 12 fits the acquired positions of the plurality of measurement target sites to a circle. For example, the calculation unit 12 fits a circle to the positions of the plurality of measurement target sites by using the least squares method. For example, the calculation unit 12 may fit, to a circle, the position information pertaining to three consecutive measurement target sites among the plurality of measurement target sites. In this case, the calculation unit 12 calculates the radius and the center position of the circle fitted to three consecutive measurement target sites. Then, for the plurality of measurement target sites, the calculation unit 12 averages the radius of the circle fitted for each group of three consecutive measurement target sites.

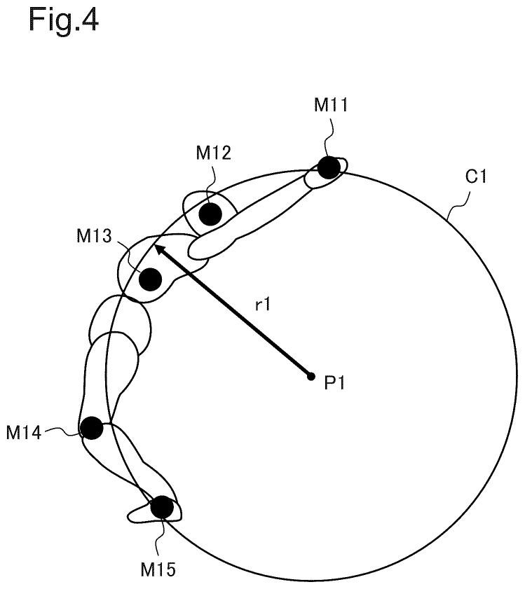

FIG. 4 illustrates an example in which a circle is fitted in a posture (extension) in which the upper body is bent backward. The calculation unit 12 fits a circle with respect to the measurement target sites M11 to M15. In the case of the example of FIG. 4, the radius of the circle fitted to the measurement target sites M11 to M15 is r1. The center position of the circle fitted to the measurement target sites M11 to M15 is P1. For example, for three groups of the measurement target sites M11 to M13, M12 to M15, and M13 to M15, the calculation unit 12 may average the fitted circles, and calculate the radius and the center position of the averaged circle.

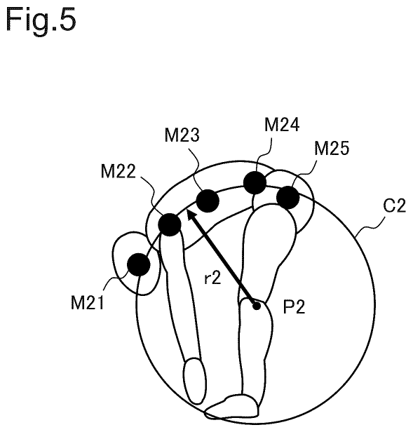

FIG. 5 illustrates an example in which a circle is fitted in a posture (bending) in which the upper body is bent forward. The calculation unit 12 fits a circle with respect to the measurement target sites M21 to M25. In the case of the example of FIG. 5, the radius of the circle fitted to the measurement target sites M21 to M25 is r2. The center position of the circle fitted to the measurement target sites M21 to M25 is P2. For example, for three groups of the measurement target sites M21 to M23, M22 to M25, and M23 to M25, the calculation unit 12 may average the fitted circles, and calculate the radius and the center position of the averaged circle.

For example, the calculation unit 12 calculates the radius r of the circle to be fitted to the plurality of measurement target sites by using following Formula 1.

$${( {X}_{i} - {X}_{0} )}^{2} + {( {Y}_{i} - {Y}_{0} )}^{2} = {r}^{2} \qquad ( 1 )$$

For example, the calculation unit 12 calculates the center position (X₀, Y₀) and the radius r of a circle fitted to a plurality of measurement target sites by substituting the positions (Xᵢ, Yᵢ) of the plurality of measurement target sites into Formula 1 and analytically solving Formula 1. For example, for the plurality of measurement target sites, the calculation unit 12 calculates the center position (X₀, Y₀) and the radius r of the circle by using the least squares method.

The calculation unit 12 calculates the curvature of the circle fitted to the plurality of measurement target sites, by using the radius of the circle. The calculation unit 12 outputs the calculated curvature of the circle to the evaluation unit 13. In a case where the radius of the circle fitted to the plurality of measurement target sites is used to evaluate the flexibility of the evaluation target segment, the calculation unit 12 outputs the calculated radius of the circle to the evaluation unit 13. For example, the calculation unit 12 may calculate the diameter as the diameter of the circle fitted to the plurality of measurement target sites.

For example, the calculation unit 12 calculates a curvature c of the circle fitted to the measurement target site by using following Formula 2.

$$c = 1 / r \qquad ( 2 )$$

The radius and curvature of the circle calculated by the calculation unit 12 are results of circle fitting.

For example, the calculation unit 12 may calculate the radius and curvature of the fitting circle by the azimuth difference or the second derivative of the position information. For example, the calculation unit 12 may calculate the radius and curvature of the fitting circle by approximating the second derivative by the Runge-Kutta method. A method of calculating the radius and the curvature of the fitting circle by the calculation unit 12 is not particularly limited.

The evaluation unit 13 acquires the result of the circle fitting by the calculation unit 12. The evaluation unit 13 evaluates the flexibility of the evaluation target segment according to the acquired result of the circle fitting. For example, the evaluation unit 13 evaluates the flexibility of the evaluation target segment according to a relationship between a preset threshold and the curvature. For example, the evaluation unit 13 evaluates the flexibility of the evaluation target segment according to a relationship between a preset threshold and the radius. For example, the evaluation unit 13 may evaluate the flexibility of the evaluation target segment according to a relationship between a preset threshold and the diameter. The evaluation unit 13 outputs the evaluation result relating to the flexibility of the subject to the output unit 15.

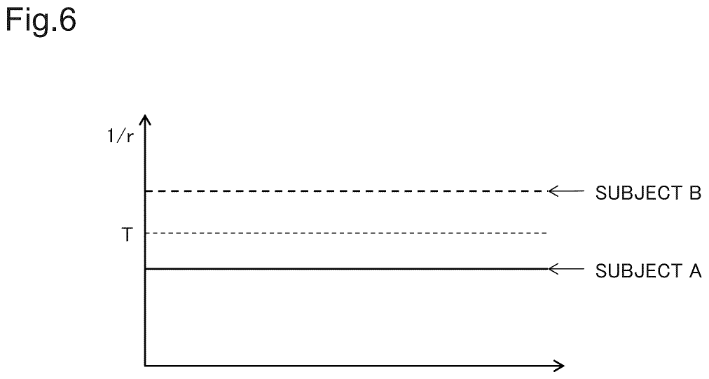

FIG. 6 is a graph illustrating an evaluation example of the flexibility of the evaluation target segment by the evaluation unit 13. A threshold T is set for the flexibility. When the curvature of the circle fitted to the bending posture of the subject exceeds the threshold T, the evaluation unit 13 evaluates that the flexibility of the subject is low. When the curvature of the circle fitted to the bending posture of the subject falls below the threshold T, the evaluation unit 13 evaluates that the flexibility of the subject is high. In the case of the example of FIG. 6, the subject A falls below the threshold T and thus has high flexibility. On the other hand, the subject B exceeds the threshold T, and thus has low flexibility. For example, the evaluation unit 13 may evaluate the flexibility according to the radius of the circle fitted to the bending posture of the subject. In that case, the evaluation unit 13 evaluates that the larger the radius of the fitted circle, the lower the flexibility, and evaluates that the smaller the radius of the fitted circle, the higher the flexibility.

The output unit 15 acquires the evaluation result of the evaluation unit 13. The output unit 15 outputs the acquired evaluation result. For example, the output unit 15 displays the evaluation result of the flexibility on the screen of the mobile terminal of the subject (user). For example, the output unit 15 outputs the evaluation result to an external system or the like that uses the evaluation result. The use of the information relating to the flexibility output from the output unit 15 is not particularly limited.

For example, the evaluation device 10 is connected to an external system or the like built in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the evaluation device 10 is connected to the mobile terminal via a wire such as a cable. For example, the evaluation device 10 is connected to the mobile terminal via wireless communication. For example, the evaluation device 10 is connected to the mobile terminal via a wireless communication function (not illustrated) conforming to a standard such as Bluetooth (registered trademark) or WiFi (registered trademark). Note that the communication function of the evaluation device 10 may conform to a standard other than Bluetooth (registered trademark) or WiFi (registered trademark). The evaluation result relating to the flexibility may be used by an application installed in the mobile terminal. In that case, the mobile terminal executes processing using the evaluation result relating to the flexibility by application software or the like installed in the mobile terminal.

### (Operation)

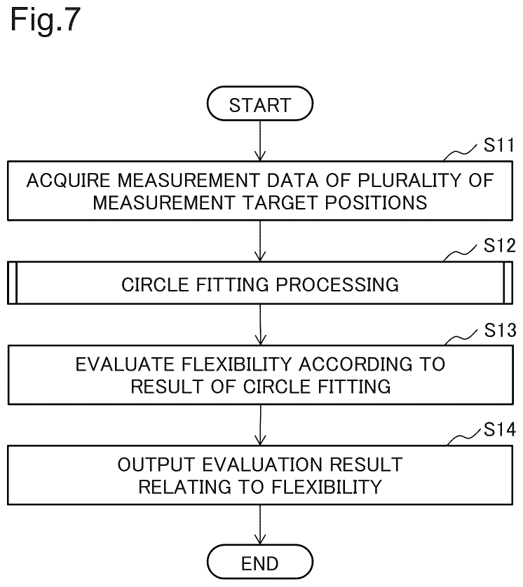

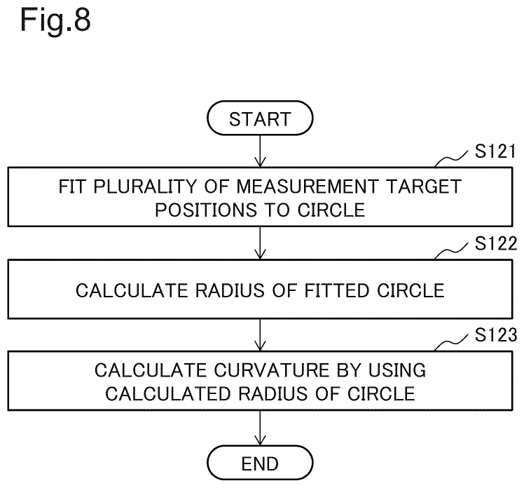

Next, the operation of the evaluation device 10 will be described with reference to the drawings. Hereinafter, the overall operation (FIG. 7) of the evaluation device 10 will be described, and then the circle fitting processing (FIG. 8) included in the overall operation will be described.

FIG. 7 is a flowchart for explaining an example of the operation of the evaluation device. In the description along the flowchart of FIG. 7, the evaluation device 10 will be described as a main operation body.

In FIG. 7, first, the evaluation device 10 acquires measurement data of a plurality of measurement target sites relating to the subject (step S11).

Next, the evaluation device 10 executes circle fitting processing (step S12). Details of the circle fitting processing in step S12 will be described later.

Next, the evaluation device 10 evaluates the flexibility of the subject according to the result of the circle fitting (step S13). For example, the evaluation device 10 evaluates the flexibility of the subject according to a relationship with a preset threshold.

Next, the evaluation device 10 outputs an evaluation result relating to the flexibility (step S14).

### [Circle Fitting Processing]

Next, the circle fitting processing (step S12 in FIG. 7) will be described. FIG. 8 is a flowchart for explaining the circle fitting processing. In the description along the flowchart of FIG. 8, the evaluation device 10 will be described as a main operation body.

In FIG. 8, first, the evaluation device 10 fits the plurality of measurement target sites to a circle (step S121). For example, the evaluation device 10 fits the plurality of measurement target sites to a circle by using the least squares method.

Next, the evaluation device 10 calculates the radius of the circle fitted to the plurality of measurement target sites (step S122).

Next, the evaluation device 10 calculates the curvature of the circle fitted to the plurality of measurement target sites by using the calculated radius of the circle (step S123). The circle fitting result such as the calculated radius and curvature of the circle is used in step S13 in FIG. 7.

### Application Example

Next, an application example according to the present example embodiment will be described with reference to the drawings. In the following application example, an example in which the evaluation result of the flexibility relating to the subject is displayed on the screen of the mobile terminal carried by the subject will be described.

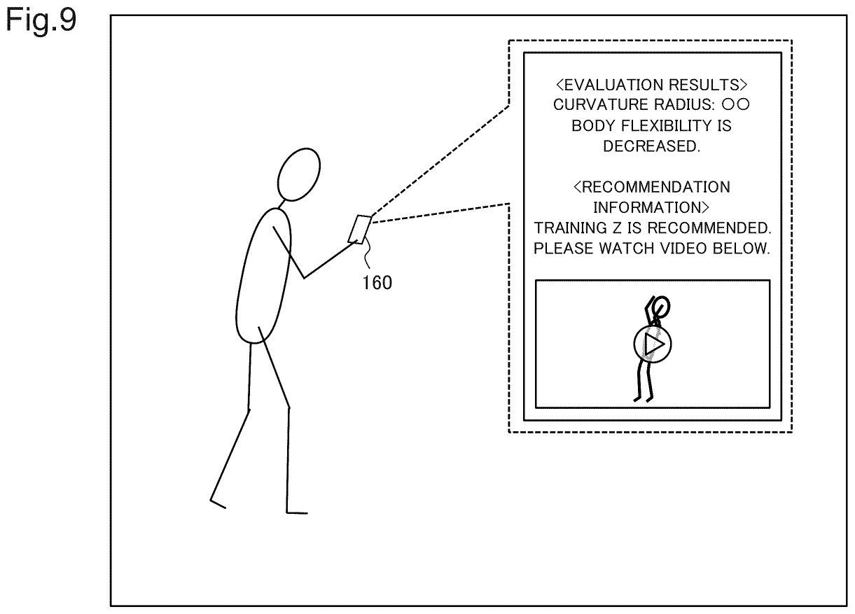

FIG. 9 is a conceptual diagram illustrating an example in which the evaluation result by the evaluation device 10 is displayed on a screen of a mobile terminal 160 carried by the subject. In the example of FIG. 9, the circle fitting result of “curvature radius: ∘∘” is displayed on the screen of the mobile terminal 160 as the information relating to the flexibility of the subject. In addition, according to the circle fitting result, information that “body flexibility is decreased” is displayed on the screen of the mobile terminal 160. Furthermore, in the example of FIG. 9, recommendation information according to the flexibility evaluation result such as “Training Z is recommended. Please watch video below.” is displayed on the display unit of the mobile terminal 160 according to the flexibility evaluation result. The user who has confirmed the information displayed on the display unit of the mobile terminal 160 can practice training leading to improvement of flexibility by exercising with reference to the video of the training Z according to the recommendation information. For example, the information relating to the flexibility evaluation result may be output to a terminal device (not illustrated) used by a trainer, a physical therapist, a family member of the subject, or the like that manages the physical condition of the subject. For example, the information relating to the flexibility may be recorded in a database (not illustrated) constructed for the purpose of health management or the like.

As described above, the evaluation device according to the present example embodiment includes an acquisition unit, a calculation unit, an evaluation unit, and an output unit. The acquisition unit acquires position data pertaining to a plurality of measurement target sites established in the evaluation target segment of the body. The calculation unit fits a circle to the plurality of measurement target sites and calculates a value of a diameter of the fitted circle. The evaluation unit evaluates the flexibility of the evaluation target segment according to the calculated value of the diameter of the circle. The output unit outputs an evaluation result relating to the flexibility of the evaluation target segment.

According to the present example embodiment, the flexibility of the body can be evaluated according to the value of the diameter of the circle fitted to the plurality of measurement target sites.

In one aspect of the present example embodiment, the calculation unit calculates a curvature before fitting to the plurality of measurement target sites. The evaluation unit evaluates the flexibility of the evaluation target segment according to the value of the curvature of the circle. According to the present aspect, the flexibility of the body can be evaluated according to the value of the curvature of the circle fitted to the plurality of measurement target sites.

In one aspect of the present example embodiment, the acquisition unit acquires the position data pertaining to the plurality of measurement target sites established in the evaluation target segment included in the spinal column of the subject in the extended posture. For the subject in the extended posture, the evaluation unit evaluates the flexibility of the evaluation target segment included in the spinal column and the uniformity in bending of the evaluation target segment included in the spinal column. According to the present aspect, the flexibility of the evaluation target segment included in the spinal column can be evaluated for the subject in the extended posture.

In one aspect of the present example embodiment, the acquisition unit acquires position data pertaining to the plurality of measurement target sites established in the evaluation target segment included in the spinal column of the subject in the forward bending posture. For the subject in the forward bending posture, the evaluation unit evaluates the flexibility of the evaluation target segment included in the spinal column and the uniformity in bending of the evaluation target segment included in the spinal column. According to the present aspect, the flexibility of the evaluation target segment included in the spinal column can be evaluated for the subject in the forward bending posture.

In one aspect of the present example embodiment, the output unit is connected to a terminal device having a screen. The output unit causes the terminal device to output information relating to the evaluation result relating to the evaluation target segment. According to the present aspect, the information relating to the evaluation result can be displayed on the screen of the terminal device. The flexibility of the evaluation target segment and the uniformity in bending of the evaluation target segment can be recognized with reference to the information displayed on the screen of the terminal device.

### Second Example Embodiment

Next, an evaluation device according to a second example embodiment will be described with reference to the drawings. The evaluation device of the present example embodiment fits the measurement target site of the subject to an ellipse and evaluates the uniformity of the bent body.

### (Configuration)

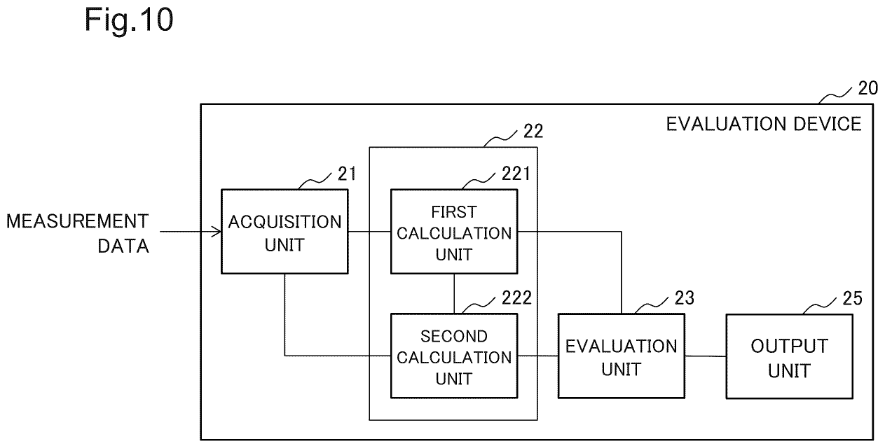

FIG. 10 is a block diagram for explaining an example of a configuration of an evaluation device 20 according to the present example embodiment. The evaluation device 20 includes an acquisition unit 21, a calculation unit 22, an evaluation unit 23, and an output unit 25. The calculation unit 22 includes a first calculation unit 221 and a second calculation unit 222.

The acquisition unit 21 has the same configuration as the acquisition unit 11 of the first example embodiment. The acquisition unit 21 acquires measurement data including the position information pertaining to the plurality of measurement target sites set in the body.

The first calculation unit 221 has the same configuration as the calculation unit 12 of the first example embodiment. The first calculation unit 221 acquires, from the acquisition unit 21, the position information pertaining to the measurement target site relating to the subject. The first calculation unit 221 fits the acquired positions of the measurement target sites to a circle.

The second calculation unit 222 acquires, from the acquisition unit 21, the position information pertaining to the measurement target site relating to the subject. In addition, the second calculation unit 222 acquires the radius of the circle (also referred to as a fitting circle) calculated by the first calculation unit 221 and fitted to the plurality of measurement target sites. Similarly to the first calculation unit 221, the second calculation unit 222 selects three consecutive measurement target sites from among the plurality of measurement target sites. The second calculation unit 222 fits the position of the measurement target sites to an ellipse for each group including the selected three measurement target sites. The second calculation unit 222 fits the positions of the three grouped measurement target sites to the ellipse, based on the positions of the three grouped measurement target sites and the radius of the fitting circle. In the ellipse fitting, the second calculation unit 222 sets the radius of the fitting circle to the long radius of the ellipse. The second calculation unit 222 calculates the short radius of the ellipse in which the radius of the fitting circle is set to the long radius. Note that the second calculation unit 222 may set the radius of the fitting circle to the short radius of the ellipse and calculate the long radius of the ellipse in which the radius of the fitting circle is set to the short radius. The second calculation unit 222 outputs, to the evaluation unit 23, the short radius of the ellipse calculated for each group. In addition, the second calculation unit 222 may fit an ellipse with respect to a group including four measurement target sites. In that case, it is sufficient if the second calculation unit 222 may fit the ellipse by using the least squares method.

For example, the second calculation unit 222 sets the radius of the fitting circle to a long radius a using following Formula 3, and calculates a short radius b of the ellipse for each group.

$${\{ \frac{( {X}_{i} - {X}_{0} ) cos θ + ( {Y}_{i} - {Y}_{0} ) sin θ}{a} \}}^{2} + {\{ \frac{- ( {X}_{i} - {X}_{0} ) sin θ + ( {Y}_{i} - {Y}_{0} ) cos θ}{b} \}}^{2} = 1 \qquad ( 3 )$$

In Formula 3 above, θ is the inclination of the long radius of the ellipse with respect to an x axis.

For example, the second calculation unit 222 substitutes the positions (Xᵢ, Yᵢ) of the three measurement target sites and the radius (long radius a) of the fitting circle into Formula 3, and calculates the center position (X₀, Y₀), an inclination θ, and the short radius b of the ellipse by using the least squares method. For example, the second calculation unit 222 may calculate the center position (X₀, Y₀), the inclination θ, and the short radius b of the ellipse by applying the least squares method to four or more measurement target sites. For example, an initial value may be set as the inclination θ.

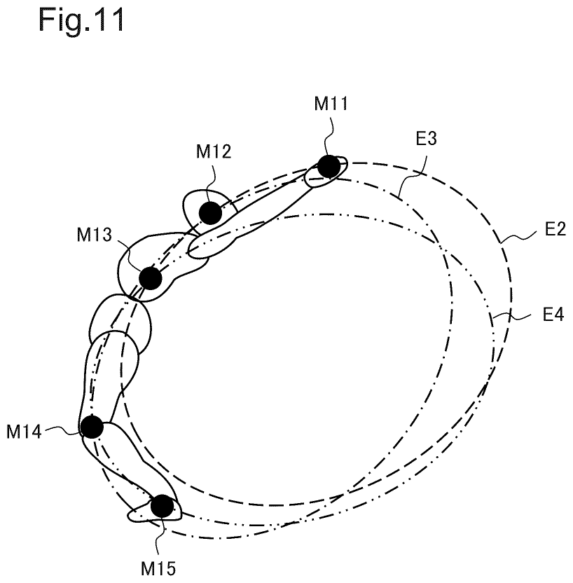

FIG. 11 illustrates an example in which the ellipse for each group is fitted to a posture (extension) in which the upper body is bent backward. The second calculation unit 222 fits ellipses with respect to three groups of the measurement target sites M11 to M13, M12 to M15, and M13 to M15. The calculation unit 12 calculates the short radii of the fitted ellipses for the three groups of the measurement target sites M11 to M13, M12 to M15, and M13 to M15. In FIG. 11, ellipses E2 to E4 are fitted with respect to the three groups of the measurement target sites M11 to M13, M12 to M15, and M13 to M15. In the case of the example of FIG. 11, the short radii of the fitted ellipses E2 to E4 are calculated for the three groups. The ellipse E2 is used for evaluating the measurement target site M12. The ellipse E3 is used for evaluating the measurement target site M13. The ellipse E4 is used for evaluating the measurement target site M14. For example, in a case where the short radius of the ellipse E3 indicates an abnormal value as compared with the short radii of the ellipse E2 and the ellipse E4, it is estimated that there is an abnormality in the measurement target site M13 relevant to the ellipse E3. The ellipse fitting can also be applied to a posture in which the upper body is bent forward (bending) or a posture in which the upper body is bent sideways (side bending). In addition, the ellipse fitting may also be performed for each evaluation target segment, such as a spinal column.

The evaluation unit 23 acquires the result of the circle fitting by the first calculation unit 221. The evaluation unit 23 evaluates the flexibility of the evaluation target segment according to the acquired result of the circle fitting. For example, the evaluation unit 23 evaluates the flexibility of the evaluation target segment according to a relationship between a preset threshold and the curvature.

In addition, the evaluation unit 23 acquires the result of the ellipse fitting by the second calculation unit 222. The evaluation unit 23 evaluates the uniformity in bending of the evaluation target segment according to the acquired result of the ellipse fitting. The evaluation unit 23 evaluates the uniformity in bending of the evaluation target segment according to the distribution of the short radius of the ellipse fitted for each group. The evaluation unit 23 outputs, to the output unit 25, the evaluation result relating to the flexibility of the evaluation target segment of the subject and the uniformity at the time of bending.

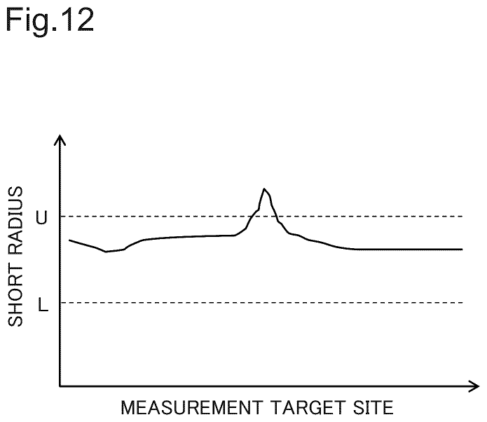

FIG. 12 is a graph for explaining the uniformity at the time of bending of the evaluation target segment by the evaluation unit 23. FIG. 12 illustrates an evaluation result relating to a certain subject. The horizontal axis of the graph of FIG. 12 indicates the measurement target site in the evaluation target segment. The vertical axis of the graph of FIG. 12 is the short radius of the ellipse fitted for each group. The short radius of the ellipse indicates an upper limit threshold U and a lower limit threshold L of the short radius of the fitted ellipse. The measurement target site of which the short radius exceeds the upper limit threshold U protrudes as compared with other measurement target sites. The measurement target site of which the short radius is less than the lower limit threshold L is recessed as compared with other measurement target sites.

In the case of the example of FIG. 12, the short radius (solid line) of the fitted ellipse for the subject exceeds the upper limit threshold U. Therefore, the evaluation target segment of the subject includes an abnormality in which a part protrudes. For example, in a case where the evaluation target segment is the spinal column, it is estimated that there is an abnormality in the bone of the measurement target site where the abnormality appears.

The output unit 25 has the same configuration as the output unit 15 of the first example embodiment. The output unit 25 acquires the evaluation result of the evaluation unit 23. The output unit 25 outputs the acquired evaluation result. For example, the output unit 25 displays the evaluation result of the flexibility of the evaluation target segment or the uniformity at the time of bending on the screen of the mobile terminal of the subject (user). For example, the output unit 25 outputs the evaluation result to an external system or the like that uses the evaluation result. The use of the information relating to the flexibility output from the output unit 25 is not particularly limited.

### (Operation)

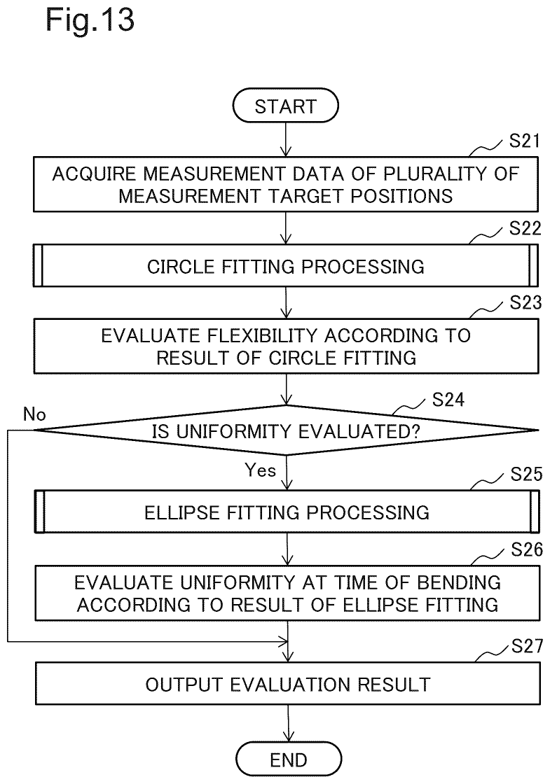

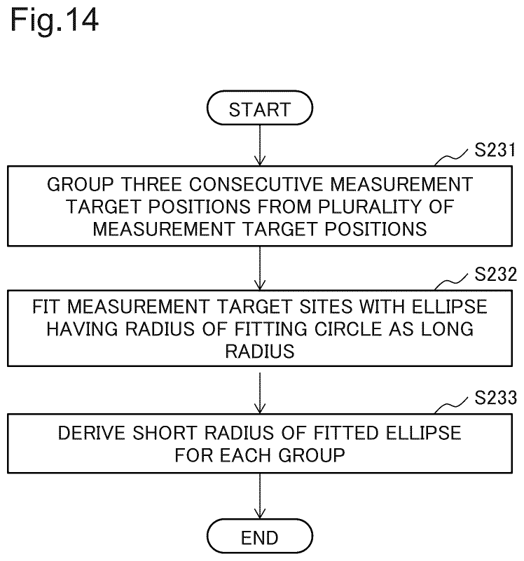

Next, the operation of the evaluation device 20 will be described with reference to the drawings. Hereinafter, the overall operation (FIG. 13) of the evaluation device 20 will be described, and then the ellipse fitting processing (FIG. 14) included in the overall operation will be described. The circle fitting processing is similar to that of the first example embodiment (FIG. 8), and thus description thereof is omitted.

FIG. 13 is a flowchart for explaining an example of the operation of the evaluation device. In the description along the flowchart of FIG. 13, the evaluation device 20 will be described as a main operation body.

In FIG. 13, first, the evaluation device 20 acquires measurement data of a plurality of measurement target sites relating to the subject (step S21).

Next, the evaluation device 20 executes circle fitting processing (step S22). The details of the circle fitting processing in step S22 are the same as those in the first example embodiment (FIG. 8), and thus the description thereof is omitted.

Next, the evaluation device 20 evaluates the flexibility of the subject according to the result of the circle fitting (step S23). For example, the evaluation device 20 evaluates the flexibility of the subject according to a relationship with a preset threshold. In a case where only the uniformity at the time of bending of the evaluation target segment is evaluated, step S23 may be omitted.

In a case where the uniformity at the time of bending of the evaluation target segment is evaluated (Yes in step S24), the evaluation device 20 executes ellipse fitting processing. Details of the ellipse fitting processing in step S24 will be described later. In a case where the uniformity at the time of bending of the evaluation target segment is not evaluated (No in step S24), the process proceeds to step S27.

Next to step S25, the evaluation device 20 evaluates the uniformity at the time of bending of the evaluation target segment according to the result of the ellipse fitting (step S26).

In the case of No in step S24 or after step S26, the evaluation device 20 outputs the evaluation result relating to the flexibility of the evaluation target segment and the uniformity at the time of bending of the evaluation target segment (step S27).

### [Ellipse Fitting Processing]

Next, the ellipse fitting processing (step S25 in FIG. 13) will be described. FIG. 14 is a flowchart for explaining the ellipse fitting processing. In the description along the flowchart of FIG. 14, the evaluation device 20 will be described as a main operation body.

In FIG. 14, first, the evaluation device 20 groups three consecutive measurement target sites from a plurality of measurement target sites (step S231).

Next, for each group of three measurement target sites, the evaluation device 20 fits the measurement target sites with an ellipse having a radius of a fitting circle as a long radius (step S232).

Next, for each group of three measurement target sites, the evaluation device 20 calculates the short radius of the fitted ellipse (step S233).

### Application Example

Next, an application example according to the present example embodiment will be described with reference to the drawings. In the following application example, an example in which the evaluation result of the uniformity at the time of bending of the evaluation target segment relating to the subject is displayed on the screen of the mobile terminal carried by the subject will be described.

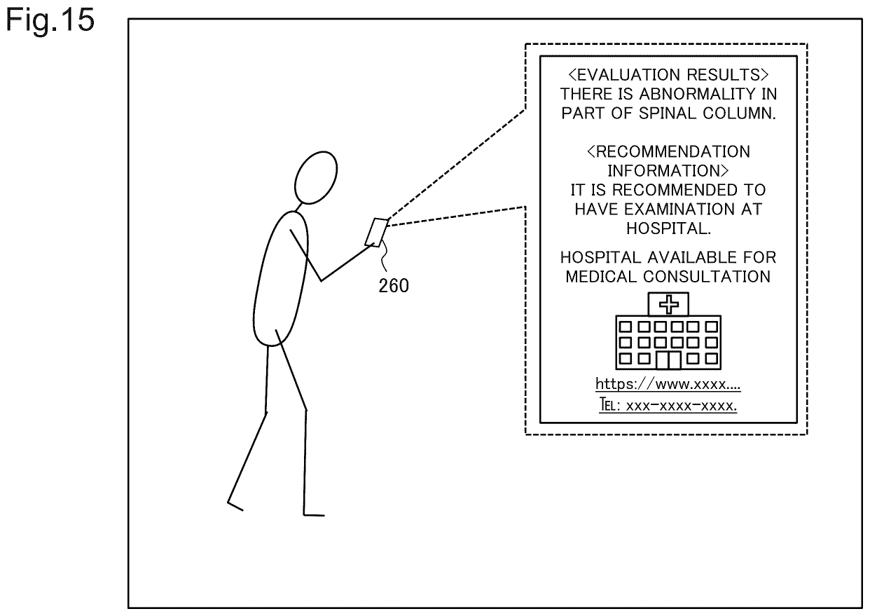

FIG. 15 is a conceptual diagram illustrating an example in which the evaluation result by the evaluation device 20 is displayed on a screen of a mobile terminal 260 carried by the subject. In the example of FIG. 15, on the screen of the mobile terminal 260, information that “there is an abnormality in a part of the spinal column” is displayed as the information relating to the uniformity at the time of bending of the spinal column that is the evaluation target segment of the subject. In addition, in the example of FIG. 15, recommendation information that “it is recommended to have an examination at a hospital” is displayed on the screen of the mobile terminal 260 according to the evaluation result of the uniformity in bending of the spinal column. Furthermore, in the example of FIG. 15, a link destination to a site or a telephone number of a hospital available for medical consultation is displayed on the screen of the mobile terminal 260 according to the evaluation result of the uniformity in bending of the spinal column. For example, the subject who has browsed the information displayed on the screen of the mobile terminal 260 and corresponding to the evaluation result of the uniformity in bending of the spinal column can select an action corresponding to the information. For example, the information relating to the evaluation result by the evaluation device 20 may be output to a terminal device (not illustrated) used by a trainer, a physical therapist, a family member of the subject, or the like that manages the physical condition of the subject. For example, the evaluation result by the evaluation device 20 may be recorded in a database (not illustrated) constructed for the purpose of health management or the like.

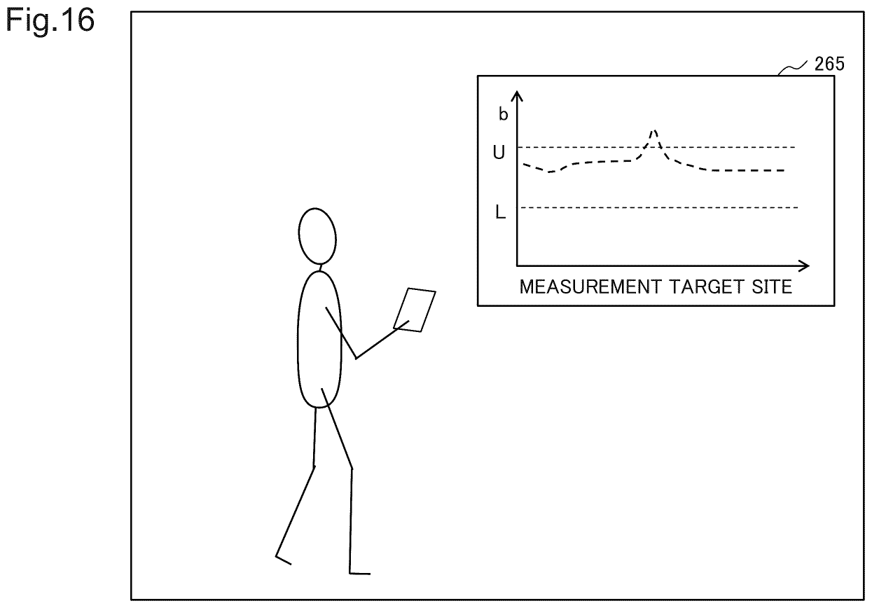

FIG. 16 is a conceptual diagram illustrating an example in which the evaluation result by the evaluation device 20 is displayed on a screen of a terminal device 265 browsable by a doctor who examines the subject. In the example of FIG. 16, a graph relating to the uniformity at the time of bending of the spinal column that is the evaluation target segment of the subject is displayed on the screen of the terminal device 265. For example, the subject who has browsed the graph displayed on the screen of the terminal device 265 and relating to the uniformity in bending of the spinal column of the subject can make a diagnosis according to the state of the subject read from the graph.

As described above, the evaluation device according to the present example embodiment includes an acquisition unit, a calculation unit, an evaluation unit, and an output unit. The acquisition unit acquires position data pertaining to a plurality of measurement target sites established in the evaluation target segment of the body. The calculation unit fits a circle to the plurality of measurement target sites and calculates a value of a diameter of the fitted circle. The calculation unit groups at least three consecutive measurement target sites among the plurality of measurement target sites. The calculation unit fits an ellipse to each group of the measurement target sites. The calculation unit calculates at least one of a short radius or a long radius of the ellipse fitted to each group of the measurement target sites. The evaluation unit evaluates the flexibility of the evaluation target segment according to the calculated value of the diameter of the circle. The evaluation unit evaluates the uniformity in bending of the evaluation target segment according to the distribution of the value of at least one of the short radius or the long radius of the ellipse fitted to each group of the measurement target sites. The output unit outputs an evaluation result relating to the flexibility of the evaluation target segment. The output unit outputs an evaluation result relating to the uniformity in bending of the evaluation target segment.

According to the present example embodiment, the uniformity in bending of the evaluation target segment can be evaluated according to the distribution of the value of at least one of the short radius or the long radius of the ellipse fitted to each group of the measurement target sites.

In one aspect of the present example embodiment, the calculation unit fits an ellipse, that is fitted to the plurality of measurement target sites and has the radius of the circle as a long radius, to each group of the measurement target sites. The calculation unit calculates the short radius of the fitted ellipse to each group of the measurement target sites. According to the present aspect, the ellipse that is fitted to the plurality of measurement target sites and has a radius of the circle as a long radius is fitted to each group of the measurement target sites, so as to make a short radius of the ellipse fitted to each group of the measurement target sites.

In one aspect of the present example embodiment, in the measurement target sites in which a value of the short radius of the ellipse fitted to each group of the measurement target sites exceeds a predetermined threshold, the evaluation unit evaluates that there is an abnormality. According to the present aspect, the abnormality of the measurement target site can be detected according to the value of the short radius of the ellipse fitted to each group of the measurement target sites.

### Third Example Embodiment

Next, an evaluation device according to a third example embodiment will be described with reference to the drawings. The evaluation device of the present example embodiment has a configuration in which the evaluation devices according to the first and second example embodiments are simplified.

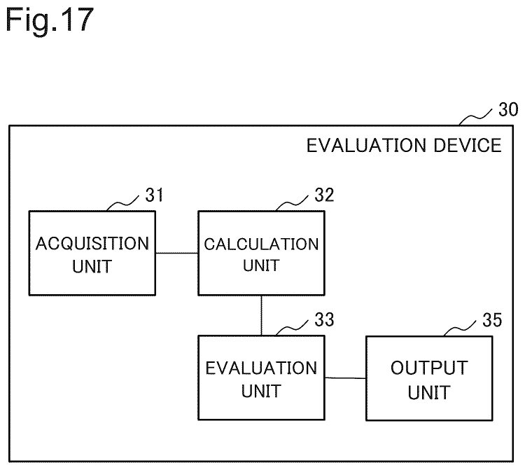

FIG. 17 is a block diagram illustrating an example of a configuration of an evaluation device 30 according to the present example embodiment. The evaluation device 30 includes an acquisition unit 31, a calculation unit 32, an evaluation unit 33, and an output unit 35.

The acquisition unit 31 acquires position data pertaining to a plurality of measurement target sites established in the evaluation target segment of the body. The calculation unit 32 fits a circle to the plurality of measurement target sites and calculates a value of a diameter of the fitted circle. The evaluation unit 33 evaluates the flexibility of the evaluation target segment according to the calculated value of the diameter of the circle. The output unit 35 outputs an evaluation result relating to the flexibility of the evaluation target segment.

According to the present example embodiment, the flexibility of the body can be evaluated according to the value of the diameter of the circle fitted to the plurality of measurement target sites established in the evaluation target segment of the body.

### (Hardware)

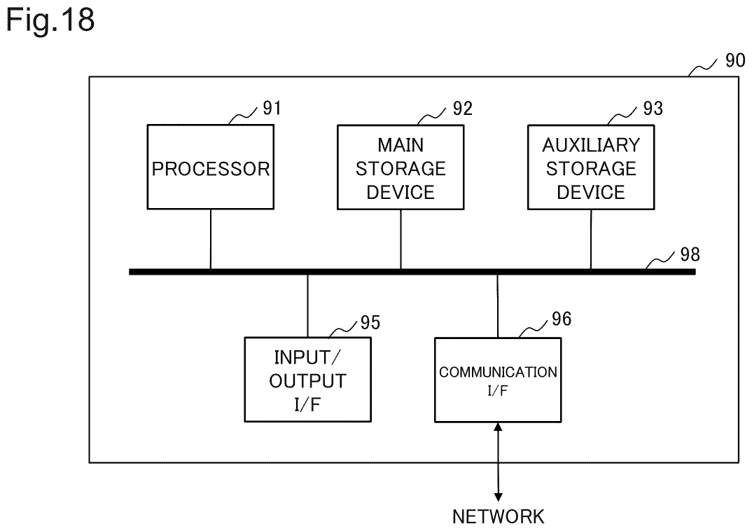

Here, a hardware configuration for executing control and processing according to each example embodiment of the present disclosure will be described by using an information processing device 90 of FIG. 18 as an example. Note that the information processing device 90 in FIG. 18 is a configuration example for executing control and processing of each example embodiment, and does not limit the scope of the present disclosure.

As illustrated in FIG. 18, the information processing device 90 includes a processor 91, a main storage device 92, an auxiliary storage device 93, an input/output interface 95, and a communication interface 96. In FIG. 18, the interface is abbreviated as an interface (I/F). The processor 91, the main storage device 92, the auxiliary storage device 93, the input/output interface 95, and the communication interface 96 are data-communicably connected to each other via a bus 98. In addition, the processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, it is sufficient if the configuration is made to use the software program installed in the information processing device 90. The processor 91 executes control and processing according to each example embodiment.

The main storage device 92 has a region in which a program is developed. A program stored in the auxiliary storage device 93 or the like is developed in the main storage device 92 by the processor 91. The main storage device 92 is achieved by a volatile memory such as a dynamic random access memory (DRAM). In addition, a nonvolatile memory such as a magnetoresistive random access memory (MRAM) may be configured/added as the main storage device 92.

The auxiliary storage device 93 stores various types of data such as a program. The auxiliary storage device 93 is achieved by a local disk such as a hard disk or a flash memory. Note that the various types of data may be stored in the main storage device 92, and the auxiliary storage device 93 may be omitted.

The input/output interface 95 is an interface for connecting the information processing device 90 and a peripheral device based on a standard or a specification. The communication interface 96 is an interface for connecting to the external system or device through a network such as the Internet or an intranet based on a standard or a specification. The input/output interface 95 and the communication interface 96 may be shared as an interface connected to an external device.

Input devices such as a keyboard, a mouse, and a touch panel may be connected to the information processing device 90 as necessary. These input devices are used to input information and settings. Note that in a case where the touch panel is used as the input device, the display screen of the display device may also serve as the interface of the input device. It is sufficient if the data communication between the processor 91 and the input device is mediated by the input/output interface 95.

The information processing device 90 may be provided with a display device for displaying information. In a case where the display device is provided, the information processing device 90 preferably includes a display control device (not illustrated) for controlling the display of the display device. It is sufficient if the display device is connected to the information processing device 90 via the input/output interface 95.

In addition, the information processing device 90 may be provided with a drive device. The drive device mediates reading of data/program from a recording medium, writing of the processing result of the information processing device 90 to the recording medium, and the like between the processor 91 and the recording medium (program recording medium). It is sufficient if the drive device is connected to the information processing device 90 via the input/output interface 95.

The above is an example of the hardware configuration for enabling control and processing according to each example embodiment of the present invention. Note that the hardware configuration of FIG. 18 is an example of the hardware configuration for executing control and processing according to each example embodiment, and does not limit the scope of the present invention. In addition, a program for causing a computer to execute control and processing according to each example embodiment is also included in the scope of the present invention. Further, a program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. In addition, the recording medium may be achieved by a magnetic recording medium such as a flexible disk, or another recording medium. In a case where the program executed by the processor is recorded in the recording medium, the recording medium corresponds to the program recording medium.

The components of each example embodiment may be arbitrarily combined. In addition, the components of each example embodiment may be achieved by software or may be achieved by a circuit.

While the invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims.

### REFERENCE SIGNS LIST

10, 20, 30 evaluation device
11, 21, 31 acquisition unit
12, 22, 32 calculation unit
13, 23, 33 evaluation unit
15, 25, 35 output unit
221 first calculation unit
222 second calculation unit

What is claimed is:

1. An evaluation device comprising:
a memory storing instructions; and
a processor connected to the memory and configured to execute the instructions to:
acquire position data pertaining to a plurality of measurement target sites established in an evaluation target segment of a body;
fit a circle to the plurality of measurement target sites and calculate a value of a diameter of the fitted circle;
evaluate flexibility of the evaluation target segment according to the calculated value of the diameter of the circle; and
output an evaluation result relating to the flexibility of the evaluation target segment.

2. The evaluation device according to claim 1, wherein
the processor is configured to execute the instructions to
calculate a curvature of the circle fitted to the plurality of measurement target sites, and
the evaluation means is configured to
evaluate the flexibility of the evaluation target segment according to a value of the curvature of the circle.

3. The evaluation device according to claim 1, wherein
the processor is configured to execute the instructions to
group at least three of the consecutive measurement target sites among the plurality of measurement target sites,
fit an ellipse to each group of the measurement target sites,
calculate at least one of a short radius or a long radius of the ellipse fitted to each group of the measurement target sites,
evaluate uniformity in bending of the evaluation target segment according to a distribution of a value of at least one of the short radius or the long radius of the ellipse fitted to each group of the measurement target sites, and
output an evaluation result relating to the uniformity in bending of the evaluation target segment.

4. The evaluation device according to claim 3, wherein
the processor is configured to execute the instructions to
fit the ellipse, which is fitted to the plurality of measurement target sites and has a radius of the circle as a long radius, to each group of the measurement target sites, and
calculate the short radius of the ellipse fitted to each group of the measurement target sites.

5. The evaluation device according to claim 3, wherein
the processor is configured to execute the instructions to
evaluate that there is an abnormality, in the measurement target sites in which a value of the short radius of the ellipse fitted to each group of the measurement target sites exceeds a predetermined threshold.

6. The evaluation device according to claim 1, wherein
the processor is configured to execute the instructions to
acquire position data pertaining to the plurality of measurement target sites established in the evaluation target segment included in a spinal column of a subject in an extended posture, and
evaluate, for the subject in the extended posture, flexibility of the evaluation target segment included in the spinal column and uniformity in bending of the evaluation target segment included in the spinal column.

7. The evaluation device according to claim 1, wherein
the processor is configured to execute the instructions to
acquire position data pertaining to the plurality of measurement target sites established in the evaluation target segment included in a spinal column of a subject in a forward bending posture, and
evaluate, for the subject in the forward bending posture, flexibility of the evaluation target segment included in the spinal column and uniformity in bending of the evaluation target segment included in the spinal column.

8. The evaluation device according to claim 1, wherein
the processor is configured to execute the instructions to
output, to a terminal device having a screen, information relating to an evaluation result relating to the evaluation target segment.

9. An evaluation method that causes a computer to:
acquire position data pertaining to a plurality of measurement target sites established in an evaluation target segment of a body;
fit a circle to the plurality of measurement target sites and calculate a value of a diameter of the fitted circle;
evaluate flexibility of the evaluation target segment according to the calculated value of the diameter of the circle; and
output an evaluation result relating to the flexibility of the evaluation target segment.

10. A non-transitory recording medium having recorded thereon a program that causes a computer to execute:
a process of acquiring position data pertaining to a plurality of measurement target sites established in an evaluation target segment of a body;
a process of fitting a circle to the plurality of measurement target sites and calculating a value of a diameter of the fitted circle;
a process of evaluating flexibility of the evaluation target segment according to the calculated value of the diameter of the circle; and
a process of outputting an evaluation result relating to the flexibility of the evaluation target segment.
